(19) United States
(12) Patent Application Publication
(10) Pub. No.: US 2018/0000651 A1
(43) Pub. Date: Jan. 4, 2018

(54) TELEMEDICAL WEARABLE SENSING SYSTEM FOR MANAGEMENT OF CHRONIC VENOUS DISORDERS

(71) Applicant: THE REGENTS OF THE UNIVERSITY OF CALIFORNIA, Oakland, CA (US)

(72) Inventors: Tingrui Pan, Woodland, CA (US); Ruya Li, Davis, CA (US); Yung-Wei Chi, El Dorado Hills, CA (US)

(73) Assignee: THE REGENTS OF THE UNIVERSITY OF CALIFORNIA, Oakland, CA (US)

(21) Appl. No.: 15/583,596

(22) Filed: May 1, 2017

Related U.S. Application Data
(63) Continuation of application No. PCT/US2015/059320, filed on Nov. 5, 2015.
(60) Provisional application No. 62/075,731, filed on Nov. 5, 2014.

(51) Int. Cl. A61F 13/06; A61B 5/0205; G01L 19/00; A61F 13/10; G06F 19/00; A61F 13/00

(52) U.S. Cl. CPC A61F 13/06; A61F 13/10; G06F 19/3418; A61B 5/02055; G01L 19/0092; A61H 2201/5058; A61H 2201/5043; A61F 2013/0094; A61H 2201/5061; A61H 2209/00; A61H 2230/06; G01L 2019/0053; A61H 2201/5082

(57) ABSTRACT

A telemedical interface pressure monitoring system is provided for intermittent or continuous monitoring of the pressure that occurs at the interface between the body and a support surface such as with a compression device, cast or resting surface. The system simultaneously measures interface pressure at multiple compression positions as well as provide real-time measurement data to both patients and clinicians. The system uses an array of one or more sensors and a data collection and transmission node with a microprocessor and transmitter/receiver that transmits the sensor data to a receiver such as a mobile device or cloud or clinic server for remote display, evaluation and automatic recording. Remote receivers can also control compression devices associated with the node.

### CROSS-REFERENCE TO RELATED APPLICATIONS

This application is a 35 U.S.C. §111(a) continuation of PCT international application number PCT/US2015/059320 filed on Nov. 5, 2015, incorporated herein by reference in its entirety, which claims priority to, and the benefit of, U.S. provisional patent application Ser. No. 62/075,731 filed on Nov. 5, 2014, incorporated herein by reference in its entirety. Priority is claimed to each of the foregoing applications.

The above-referenced PCT international application was published as PCT International Publication No. WO 2016/073777 on May 12, 2016, which publication is incorporated herein by reference in its entirety.

### STATEMENT REGARDING FEDERALLY SPONSORED RESEARCH OR DEVELOPMENT

This invention was made with Government support under 1307831, awarded by the National Science Foundation. The Government has certain rights in the invention.

### INCORPORATION-BY-REFERENCE OF COMPUTER PROGRAM APPENDIX

Appendix A referenced herein is a computer program listing in a text file entitled “UC-2015-260-2-US-appendix-A.txt” created on May 1, 2017 and having a 28 kb file size. The computer program code, which exceeds 300 lines, is submitted as a computer program listing appendix through EFS-Web and is incorporated herein by reference in its entirety.

### BACKGROUND

1. Technical Field

The present technology pertains generally to medical sensing and monitoring devices and methods and more particularly to a telemedical compression therapy system for use as a treatment of chronic venous disorder.

### 2. Background

Venous blood flow from the extremities must overcome gravity to return to the heart when the body is upright. Contractions of the muscles of the thigh, calf and foot force the venous blood upwards against the forces of gravity. Valves within the veins prevent the venous blood from flowing backward and away from the heart. The valves open again when the leg muscles contract allowing the blood to flow towards the heart.

Effective return of venous blood to the heart depends on normal muscle activity and a properly functioning venous system. The venous system in the lower leg is a network of superficial veins that are connected to deep veins in the interior of the leg by the perforator veins. If the valves of the superficial veins, perforator veins or the deep veins do not function properly, venous drainage and effective venous return to the heart are impaired. Valve failure or valve incompetence will allow the venous blood to flow back down to the previous section of the vein and ultimately reducing the return of venous blood back to the heart causing venous hypertension.

Venous valve failure can be idiopathic or caused by damage from a variety of diseases or conditions such as venous thrombosis, venous obstruction or damage due to trauma. Venous valve insufficiency can also be amplified by patient immobility, inactivity or an abnormal gait that reduce the effectiveness of the natural calf muscle pumping action. Ultimately, venous valve failure leads to venous hypertension and results in chronic venous disorder.

Chronic venous disorder can manifest as spider veins, varicose veins, various skin changes as well as venous ulcerations. As an example, venous leg ulcers (VLU) result from venous hypertension due to superficial, perforator and/or deep venous valve failure or incompetence. Venous hypertension activates the inflammatory system and changes the microvasculature which leads to the formation of a VLU and can impede the healing of the ulcer.

Venous hypertension also causes capillaries in tissues to become more permeable allowing leakage of various blood cells, proteins and fluid into surrounding tissues. This also results in reduced tissue oxygenation and microvascular abnormalities. Common secondary conditions arising from this milieu include hyperpigmentation, eczema, edema, lipodermatosclerosis, and other changes in tissue hardness and skin appearance.

As much as 50% of the adult population may suffer from some form of chronic venous insufficiency and related symptoms. For example, venous leg ulceration occurs in up to 5% of the population over 65 years of age and in 1.5% of the general population. It represents a significant health care burden in the Western hemisphere. This trend will only increase given the growth of aging population world-wide. In the United States, this translates to approximately 2 to 3 million individuals affected by the condition. More importantly, 2 million workdays per year are lost due to venous leg ulcerations and may cause early retirement and disability. Furthermore, the conservative estimated cost of treating venous leg ulceration is about $3 billion per year, a significant health care cost.

Compression therapies directed to the extremities have been developed and used to treat patients with a variety of lymphatic and venous disorders of the limbs. The aim of compression therapy is 1) to improve the velocity and flow of venous blood and lymph return 2) to decrease edema and reduce venous hypertension and its long-term complications. Compression therapies are also used to prevent chronic venous disease progression and as a prophylaxis of venous thromboembolism. The systematic use of compression therapy has been scientifically proven to be the best treatment for venous leg ulcers.

Based on published literature, compression therapy remains the main treatment modality for venous leg ulcers and has a GRADE IA recommendation from the most recent clinical practice guideline published by the Society for Vascular Surgery (SVS) and the American Venous Forum (AVF). The positive effects of compression therapy will depend on the characteristics of the pressure that is being applied and the nature and severity of the disease that is being treated.

The main methods of compression therapy are 1) compression bandages (elastic, long stretch, short stretch, inelastic) or 2) elastic compression hosiery, or 3) pneumatic compression sleeve systems. These methods may be used individually or in concert with each other. For example, a compression bandage may be used for acute treatment of a venous leg ulcer while compression hosiery can be used for maintenance once the ulcer has healed.

Compression bandage systems can be single or multi-layer (2 to 4 layer) bandages. Typically, the compression bandages are wrapped around the ankle and lower leg with overlapping turns in order to provide either a uniform pressure or a graduated pressure from ankle to calf. Inelastic bandage materials normally exert higher pressures than elastic bandages and may be selected over elastic materials for treatment of certain conditions.

Medical compression stockings are made of resilient elastic textiles and are available in standard knee-length, thigh-length or waist-high sizes. They are classified according to the exerted pressure profiles that are established by the manufacturer experimentally. The difference between the resting and working pressures that are exerted by compression garments is inversely proportional to their overall elasticity.

However, the applied compression pressure of a compression system is a dynamic parameter that changes with the activity and position of the body of the patient. Ideally, the compression device exerts a low interface resting pressure when the patient is in a horizontal position and exerts an increased interface pressure to counteract the increasing venous pressure when the patient stands up. Generally, the stiffness of the applied compression device system defines the change in interface pressure between resting and standing. Thus, when the patient stands or walks and leg muscles contract, an inelastic material will produce a higher increase in interface pressure than will be produced by an elastic material.

Although the effectiveness of a compression therapy depends on the pressure that is actually applied, current clinical treatments with bandages or compression stockings do not measure the applied (interface) pressure.

Rather, the current schemes in the art calculate an initial pressure when the bandages are applied and assume the same pressure is applied throughout the course of treatment. Specifically, the initial application pressure exerted by a compression bandage depends on the thickness (t) of the material used, the width of the bandage (w), the number of overlapping turn layers (n), the radius of the limb (R) and the applied tension (T) of the material. The relationship between these variables can be expressed by Laplace's law: P=Tnt/Rw.

A major concern with existing compression therapy systems is that the compression pressure that is actually applied by the system may vary from the interface pressure that is assumed to be applied by the system over the course of treatment. As a result, this leads to imprecise clinical treatments because the actual interface pressure dosage is not quantified.

Another source of uncertainty in compression therapy is the variation in the bandage applications between caregivers and the declining pressures produced by the bandages over time from material fatigue.

Compression bandage placement is often performed by the clinical staff. Due to the lack of consistent interface pressure measurements the interface pressure will vary each time a new bandage is placed. The experience-based wrapping by caregivers is not only arbitrary but can lead to inconsistency in interface pressure which produces variations in clinical outcomes. The level of interface pressure provided between each clinical visit for bandage change remains ambiguous and inconsistent with current therapy techniques.

A further consequence of the reliance on experience based wrapping or the classification of the compression garment instead of pressure measurements is that the overall therapy is not optimized so the course of treatment may be lengthened or even ineffective. The patient may also endure unnecessary pain and discomfort with a wrapping or compression garment that exceeds a therapeutic pressure. Published literature indicates an interface pressure between 20 to 40 mmHg is optimized for venous ulcer healing but the fact that interface pressure is rarely measured during compression therapy, the effectiveness of any given therapy to reach this therapeutic pressure range is unknown.

Physicians have no quantitative measurements to evaluate how the compression therapy has performed and patient compliance with the therapy cannot be monitored. Accordingly, there is a need for a device that can provide intermittent, continuous and consistent interface pressure measurements to the physician in clinic and to the patient or caregiver at home so that the applied (interface) pressure can be measured, the overall therapeutic pressure dosage can be quantified and patient compliance can be monitored.

### BRIEF SUMMARY

The technology described herein generally comprises a wearable pressure sensing system and a telemedical monitoring system for the management of chronic venous disorders. The system provides the ability to measure pressure dosages over the course of treatment in real time and automatically documented. Interface pressure is defined as the pressure that occurs at the interface between the body and the support surface such as with a compression device.

Current compression therapies treat the applied interface pressure as a static parameter and the duration of treatment is based on the clinical response of the patient. A therapeutic pressure dose is never characterized or determined and treatment parameters are not optimized in these therapies. However, the applied interface pressure of a compression system is a dynamic parameter that regularly changes with the activity and position of the body of the patient as well as material fatigue of the compression materials and the reduction of edema in the patient.

Accordingly, the present system allows for monitoring and recording of the actual pressure exerted by the compression device over the course of treatment. Rather than estimate an applied pressure and the pressure dosage, as done presently in the art, the system allows for the acquisition of actual interface pressures at different locations along the limb.

The response of the patient to the compression treatments over time can also be evaluated. The needed changes in pressure treatment can be made quickly to allow the overall treatment of a particular patient to be optimized. If a wearable device is available to the clinical staff each time a compression bandage therapy is performed, the bandages can be maneuvered to keep interface pressure within a certain therapeutic range determined by the physician and this will translate to objective quality assurance, decreased treatment times and patient tailored treatments.

Pressure excesses at locations along the appendage can also be identified to avoid the occurrence of pressure damage over bony or tendinous prominences of the leg, for example. Likewise, the system can verify the actual pressures at different locations on the appendage in cases where graduated compression up the leg is desired.

The system can also determine if the compression bandages or other device was applied properly and can identify any changes in applied compression that may take place over time due to material fatigue or anatomical changes etc. Traveling to and from clinical visits remains an obstacle for many patients with bulky compression bandages since such patients are often not fully ambulatory. Accurate status monitoring by the system of the compression device remotely can also eliminate the need for frequent clinical visits, so the patient can avoid taking time off from work and minimize wage loss, cost of travel and loss of time. Moreover, this will lead to a better quality of life given that such a treatment modality can be performed at the comfort of the home of the patient on a consistent basis.

The system utilizes at least one compression applicator that incorporates one or more pressure sensors that are coupled to an applicator controller. The applicator may be a compression garment, compression bandage, pneumatic sleeve or other suitable compression device for compressing sections of an appendage. The sensors are typically located at set points along the length or circumference of the appendage. For example, individual sensors may be mounted to the interior of a compression sock to properly orient the sensors on the patient. The sensor locations on the sock, for example, can also be customized to the appendage dimensions of a particular patient.

The application controller preferably has a transmitter and a microcontroller and control function that receives a signal from the sensors and processes and transmits the signal through the transmitter. The preferred transmitter is a Bluetooth, WiFi or similar format transmitter that can communicate with hand held devices such as cellular telephones or tablets as well a wireless routers and an Internet connection. The optional hand held devices can act as an interface with the sensor controller and display of the pressure and other sensor data in one embodiment. In another embodiment, the sensor data is transmitted through a WiFi link to a cloud or clinic server for recording, analysis and physician evaluation.

According to one aspect of the technology, a system is provided with sensors that have a low profile, lightweight, stable and suitable for prolonged applications on an appendage of a patient for pressure treatment of various vascular disorders.

Another aspect of the technology is to provide a system that can intermittently and/or regularly monitor the interface pressure of a compression device at desired locations on the appendages of a patient and collects and transmits sensor data to a remote location for physician analysis and automatic storage.

According to another aspect of the technology, a pressure monitoring system is provided where measurement data may be directly displayed on mobile devices of the patient or physician or remotely transmitted to the clinical offices via internet, cellular or other wireless networks.

A further aspect of the technology is to provide a system that allows remote sensing and control of an inflatable pressure device permitting temporal control over the applied pressures at specific locations along the appendage of the patient.

Another object of the technology is to provide a circulation improvement system that is lightweight, reusable or disposable that has a telemetric circuit that is miniaturized with ultralow power for extended use.

Another object of the technology is to provide a remote monitoring and control system that allows remote control over the compression device and sensors by a physician.

Further objects and aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic system diagram of a method for real time remote sensing and monitoring of compression therapy according to one embodiment of the technology.

FIG. 2A is a detail view depicting a sensor array of seven sensors and a single data collector/transmitter node according to one embodiment of the technology.

FIG. 2B is a cross-sectional view of one embodiment of a pressure sensor according to the technology.

FIG. 3A is a schematic side view of a lower leg bandage wrap embodiment with four single sensor-data collector/transmitter nodes positioned at locations on the foot, ankle, calf and thigh of the patient.

FIG. 3B is a schematic side view of a zippered compression hose on the lower leg with a single sensor-data collector/transmitter node positioned at the calf musculature of the patient.

FIG. 4A is a schematic top view of a lower arm bandage wrap embodiment with two sensor-data collector/transmitter nodes positioned at points on the forearm of the patient.

FIG. 4B is a schematic top view of a lower arm fabric compression sleeve embodiment with two sensor-data collector/transmitter nodes positioned at points on the forearm of the patient.

FIG. 4C is a schematic top view of a lower arm cast or splint compression device embodiment with two sensor-data collector/transmitter nodes positioned at points on the forearm of the patient.

FIG. 5 is a schematic view of a sensor array-data collector/transmitter node coupled to a compression device or lining according to one embodiment of the technology.

FIG. 6 is a side view of a compression wrap device embodiment secured by hook and loop fasteners with an eight sensor array and a corresponding pressure display showing applied pressures at each of the sensors.

FIG. 7 is a detail view of a display screen of a pressure plot over time from a single pressure sensor as well as indicators of network connection, battery level threshold alarm and real time pressure.

FIG. 8 is a side view of an alternative embodiment of a controllable inflatable pressure sleeve with individually inflatable rings and individual pressure sensors and the ring pressurization can be controlled remotely.

FIG. 9 is a circuit diagram of circuitry for acquiring interface pressure from a sensing array according to one embodiment of the technology.

### DETAILED DESCRIPTION

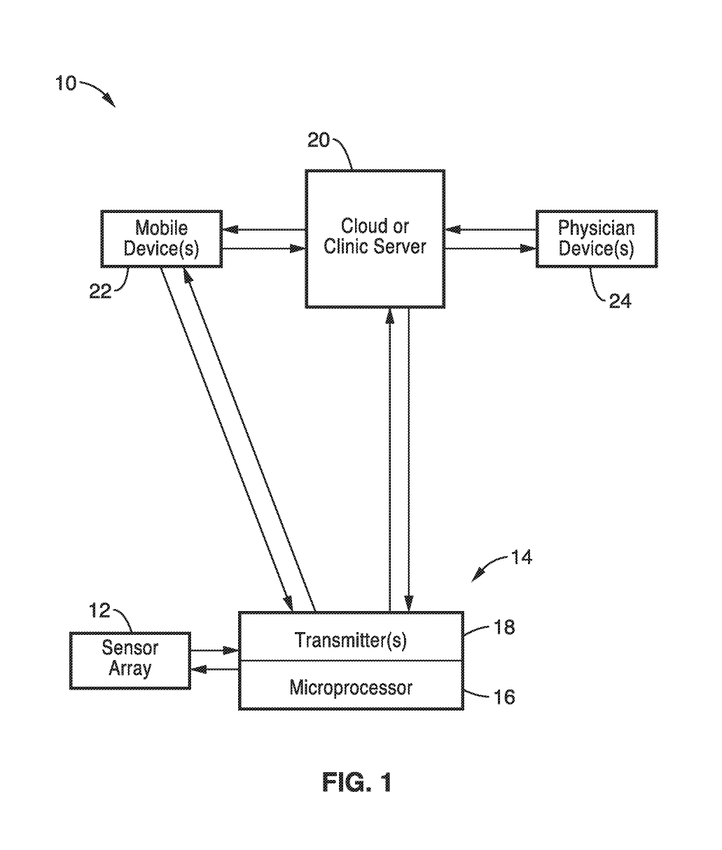

Referring more specifically to the drawings, for illustrative purposes, embodiments of the compression therapy system and methods for pressure monitoring, recording and control are generally shown. Several embodiments of the technology are described generally in FIG. 1 through FIG. 9 to illustrate the apparatus and methods. It will be appreciated that the methods may vary as to the specific steps and sequence and the apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, a monitoring and recording system 10 with a compression device with sensor array 12 and single data collector and transmitter node 14 are depicted schematically and not to scale. The illustrated system 10 generally provides a compression therapy method that intermittently or continuously monitors the interface pressure at desired locations on the appendages of a patient and collects and transmits sensor data to a remote location for physician analysis and storage. In one embodiment, the collected or streamed sensor data is transmitted to the cloud or clinic server 20, processed and automatically made part of the health records of the patient.

The pressure and other sensor data can be continuously, periodically or intermittently monitored for levels beyond set thresholds that may indicate the need for intervention by health care providers. For example, if the pressure levels drop below a designated level that is therapeutic for a set period of time, then the patient may need to return to the clinic to have the bandages re-wrapped. Similarly, if the pressure levels drop below a designated level for a set period of time, then the history will indicate that the patient is failing to comply with the therapy and an a communication with the patient is needed.

There are a number of compression therapy devices that have been shown to provide pressure on an appendage to produce a therapeutic effect on the vasculature and blood flow of a patient. Compression therapy devices include bandages, wraps, single/multilayer compression systems, compression garments, and pneumatic compression products, etc. The selection of a compression therapy product will depend on the physiological deficiency that is to be treated. However, the effectiveness of a compression device depends on the pressure that is applied, whether the device is applied properly and any changes in the actual pressure that is applied over time.

Individual sensor or an array of sensors 12 are incorporated into the compression therapy device. For example, the sensors 12 can be sewn into compression socks or adhered directly on the skin of the patient at designated locations. Data collection sensors can be attached to body using different fasteners or adhesives including Varco wrap, rubber bands, adhesive silicone, and tape, etc. The pressure sensors can also be covered by adhesive or non-adhesive dressings, bandages, pads, gauze sponges, kling wrap, etc. Additionally, the sensors can be applied over a single or multiple layers of non-compressive separation materials to measure interface pressure.

The system, through a single or multi-channel sensor array will enable monitoring of interface pressure originated from any clothing (medical and non-medical), instrument, covering, protection, and wearable over any part of the human body including but not limited to head, face, neck, ear, shoulder, back, chest, abdomen, pelvis, waist, torso, hip, and limbs. The system can also be utilized to monitor the interface pressure during the course of medical procedure or treatment, such as chronic venous disease (venous leg ulceration bandage and compression treatment), lymphedema bandage and compression treatment, burn patients bandage and compression treatment, trauma patients bandage treatment, patients with limb fractures/limb sprain requiring soft or hard cast, patients on a surgical table to identify high interface pressure points.

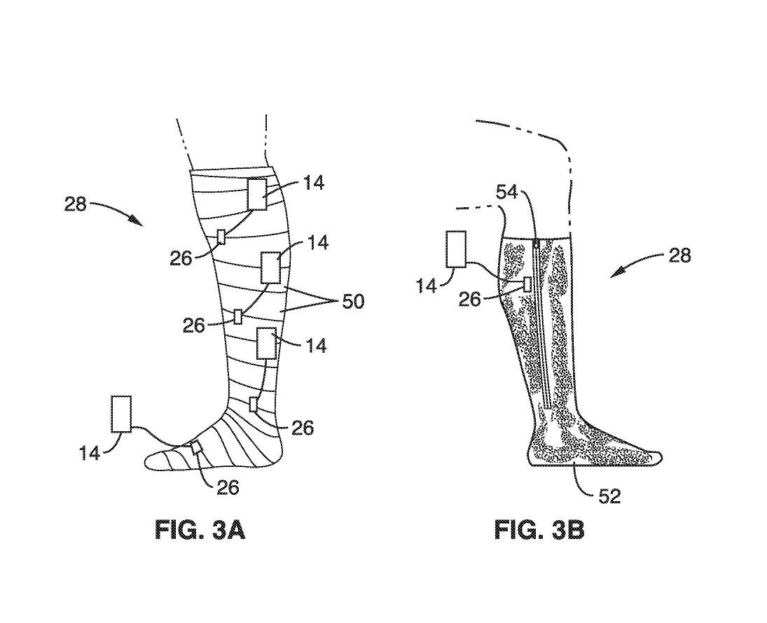

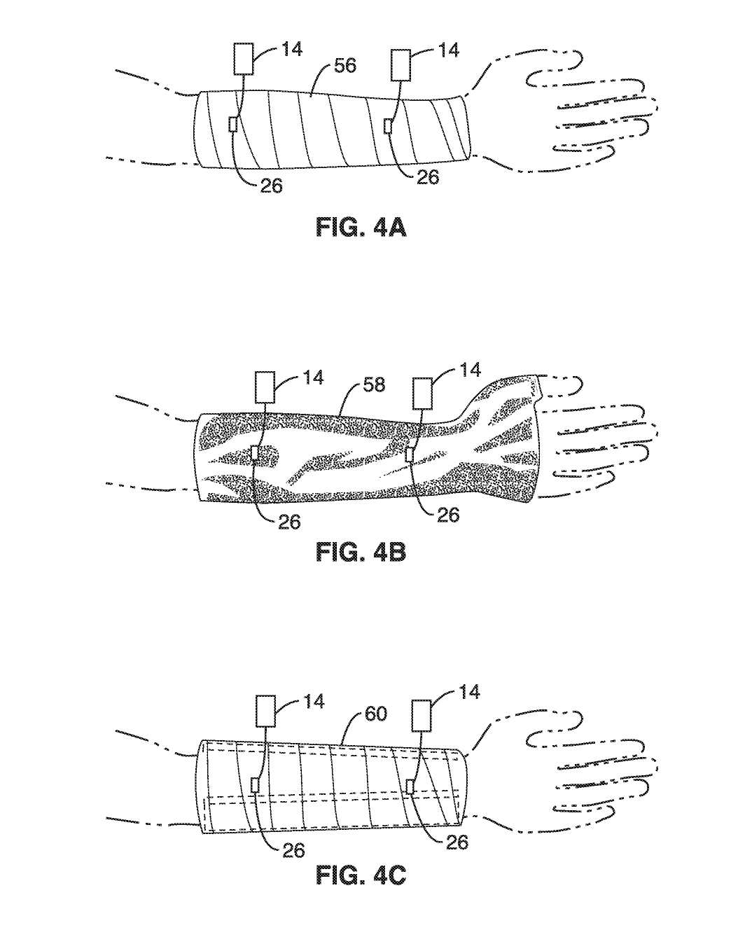

Accordingly, the sensor-data/transmitter node units can be associated with any type of compression device or setting where pressure sensing is needed. Typical adaptations include use with compression bandages 50 to an appendage such as on one or more parts of the leg as shown in FIG. 3A or with a compression stocking 52 as shown in FIG. 3B. Another typical adaptation is the placement of sensors on one or more parts of the arm or hand with compression bandages 56 as shown in FIG. 4A; with a compression sleeve 58 as shown in FIG. 4B or with a cast or splint 60 as illustrated in FIG. 4C. Although these illustrations are for devices applied to the lower arm or lower leg, it will be understood that the sensors can be used with devices applied to the whole arm or leg as well as other parts of the body.

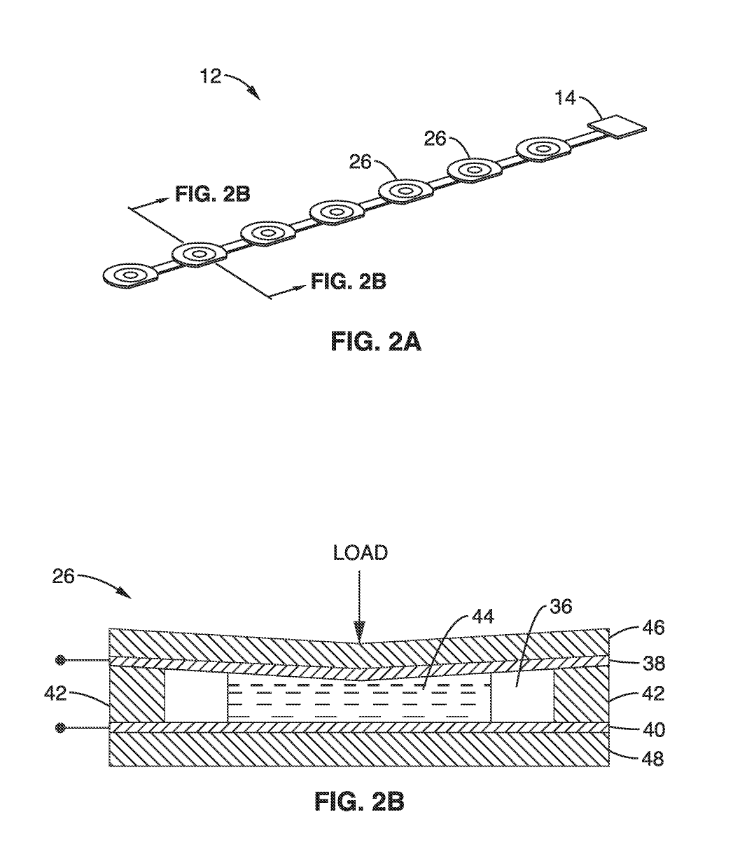

One configuration of a linear sensor array with seven sensors 26 and a data/transmitter node 14 is shown in FIG. 2A. The sensor array 12 can include multiple sensors in any configuration. The sensor array can also be a single sensors coupled to a data/transmitter node and placed individually at locations on a leg with compression bandages 50 as shown in FIG. 3A. The placement of each of the sensors 26 at select points on the leg takes place before the application of the bandages 50. The data/transmitter nodes 14 of each sensor are coordinated or transmitted separately.

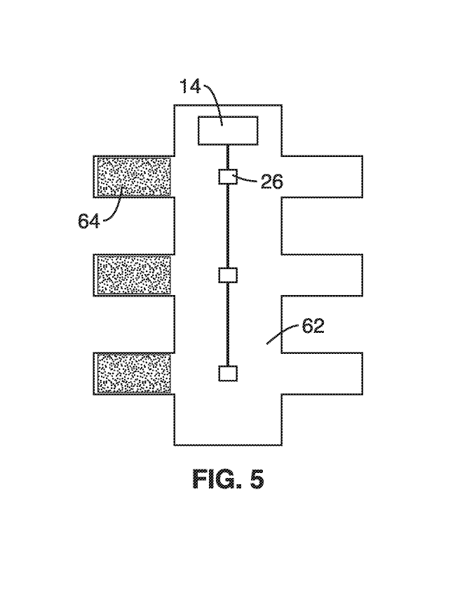

The sensor arrays 12 can also be placed at specific locations or they can be incorporated in compression garments so that the locations are pre-determined by the placement of the sensor in the garment 52 as shown in FIG. 3B and FIG. 5. The compression device 28 in FIG. 3B is a compression stocking 52 that has a zipper 54 for easy removal. In this illustration, the compression stocking 52 has a single sensor 26 and data/transmitter node 14 that is placed at the calf.

The sensor arrays and data/transmitter node can also be incorporated directly into other removable compression garments or compression liners that are part of a compression device approach. In the embodiment shown in FIG. 5, three sensors 26 and data/transmitter node 14 are mounted to the body of a compression wrap 62 or liner that is easily removable with the release of hook and loop fasteners 64.

The sensor array and data collecting/transmitter units that are associated with a selected compression therapy device 28 can have single or multiple pressure sensors and other sensors that communicate with one or more data/transmitter nodes 14. Suitable pressure sensors 26 preferably have a thin profile and made from flexible materials that can be tolerated by patients for long periods of time. For example, the flexible pressure sensor can be a force sensitive rubber (FSR) based pressure sensor, conductive ink based pressure sensor, conductive polymer based capacitive pressure sensor, or a microfluidic based pressure sensor. The resistance change of the FSR based pressure sensor can be based on conductance of the FSR change or the rubber-electrode contact area change. Other resistance or capacitance based sensors can also be used.

One particularly preferred sensor configuration is shown schematically in the cross-section of FIG. 2B. In this embodiment, a sensor 26 is a liquid-based impedance pressure sensor. The sensor 26 comprises a partially-filled or completely filled liquid chamber 36 formed by a top electrode 38 and a bottom electrode 40 separated by circular spacing wall 42. A membrane 46 is coupled to the surface of top electrode plate 38 and a support membrane 48 is coupled to the bottom electrode 40.

Chamber 36 encloses a droplet or column of liquid 44 such as an electrolyte solution in the embodiment shown in FIG. 2B. The pressure/force sensor 26 utilizes an electrical double layer (EDL) of parallel electrode layers/plates 38, 40 at the liquid/solid interface as the sensing elements.

Under external mechanical loads, one or both of the deformable membranes 46, 48 will change shape, and as a result, the contact area of the liquid-electrode interface will expand (assuming an incompressible fluid with unaltered volume of the liquid). This design can have both capacitive and resistive modes. In a capacitive mode, the variation in the contact area will lead to a proportional change in the interfacial capacitance. In a resistive mode, the displacement of the fluidic sensing layer will produce a measurable change in the resistive values existing between the two electrodes 28, 40.

The sensor embodiment of FIG. 2B is preferred because the units have been shown to have a very fast mechanical response with low-viscosity sensing units producing a response within a millisecond range and a high capacitive sensitivity of 0.45/kPa at its dimension. The sensors are also chemically and thermally stable as they are made of primarily flexible polymer materials with tunable elasticity and extensive deformability.

Referring back to FIG. 1, the sensors of the sensor array 12 communicate data with the microprocessor 16 of the data/transmitter node 14. Data collection/transmitter node 14 can be detachable from the sensing units in one embodiment. The sensors 12 can also be calibrated and controlled by and through the microprocessor 16.

An example of computer programming instructions in a derivative of Texas Instruments Keyfob Developer Kit in C programming language for performing functions on the microprocessor 16 described herein are set forth in Table 1 and Table 2 of Appendix A. The original code was obtained from examples for the CC2541 Keyfob developer kit, provided by Texas Instruments, and modified from their original contents according to embodiments of the technology described herein as follows:

(a) heartrate_Main.c—lines with functional modifications: 99-102 (initializing digital I/O and ADC configurations).

(b) heartrate.c—lines with functional modifications: Lines 101 (redefining measurement notification rate); 123 (redefining battery measurement notification rate); 181-204 (redef. device name for advertising); 219-220 (inserted data space to add periodic battery check); 225 (redef. device name for connectivity); 263, 267 (created new methods for battery check implementation); 313 (redef to start advertising when powered on); 380 (registering batt check for callbacks); 392-404 (re-init dig I/O and peripheral pins appropriately); 564-582 (input code to perform adc measurement of pressure); 748-763 (new method for battery check callback impl); and 796-806 (new method for battery check impl).

The sensors of the array 12 include at least one pressure sensor and can also include other sensors such as temperature sensors, heart beat sensors, moisture (sweat) sensors and chemical detection sensors.

The microprocessor 16 can also have a data storage capability in one embodiment. Sensor data that is acquired can be optionally stored and periodically downloaded with a wired connection during an office visit or by the patient at home. The manually downloaded data can be stored at the clinic and made part of the patient record. In another embodiment, the microprocessor has a display so that the pressure data of the sensors and unit status can be displayed in real time.

The data/transmitter node 14 has one or more transmitters 18 and receivers. In one embodiment, the transmitter/receiver 18 is configured for wireless communications (Wi-Fi) through an access point and router or other wireless communications scheme. In the configuration of FIG. 1, the transmitter 18 of node 14 can communicate with a cloud server or clinic server 20. The transmitter 18 of node 14 can also communicate with one or more mobile devices 22 through a Bluetooth, Wi-Fi, radio, 1G, 2G, 3G, 4G, RFID and Near Field Communications or other suitable wireless communications scheme. More than one type of transmitter or transmitter/receiver can be incorporated in the node 14. The mobile devices 22 can also communicate with the cloud or clinic servers 20 as shown in FIG. 1. However, this is optional.

In one embodiment, the transmitter 18 of node 14 only communicates with one or more mobile devices 22 that serve as an interface and provide data processing, display and data storage functions. In this embodiment, the mobile device 22 of the patient can display the pressure data and receive an alert if the pressures deviate from a set point. Likewise, a mobile device 22 of a physician or other healthcare worker can receive and process the sensor data.

In the embodiment shown in FIG. 1, the data from the node 14 can be received by the server 20 either directly or indirectly through a mobile device 22. The physician or other healthcare provider can have access and control with a computer, mobile or other control device 24. The physician through the control device 24 can also communicate with the mobile device 22 of the patient as well as each of the data/transmitter nodes 14 of the compression device 28 through the Bluetooth, Wi-Fi, 1G to 4G, or other wireless communications system and the transmitter/receivers 16 of each node 14. For example, the physician can message the mobile device 22 directly when the processed sensor data indicates a pressure that is outside a set threshold and intervention is necessary.

Accordingly, the system via a single or multi-channel sensor array 12 will enable physician-monitoring and self-monitoring of interface pressure originated from non-medical grade stocking, legging, socks, glove, sleeve, and supportive devices as well as medical grade compression stocking, progressive compression stockings and compression sleeves. The system can also be used to monitor interface pressure from a cast (including soft, hard, fiberglass and plaster) and splints (including soft, hard, fiberglass and synthetic).

The system 10 will also allow for self-adjustment or care-giver and/or healthcare provider adjustment of compression bandage (long and/or short), wrap, single or multilayer compression system, compression garment to obtain the targeted interface pressure range and reach clinical value by either loosening or tightening of compression device 28.

The system will also be able to track patient compliance/adherence to the therapy protocols and analysis of therapy progress. Using the number of device sensor activations per day and week, the system device can demonstrate how many days a week and/or hours per day patient is actually receiving a therapeutic dose of pressure from the prescribed compression product. Additionally, the duration of time that the patient remained under therapeutic compression can also be demonstrated and recorded.

The system not only directly measures interface pressure, it can also indirectly measure other physiological parameters such as muscle contractility (both duration and intensity), temperature and heart rate. It offers the highest pressure sensitivity and accuracy with ultrafast mechanical responses in a soft skin-like construct in addition to its convenient wireless user interface. Notably, the pressure sensing array is a viable solution capable of simultaneously measuring interface pressure at multiple compression positions as well as providing real-time measurement data to both patients and clinicians.

Processed sensor data and other relevant information can be potentially displayed on several devices in the system illustrated in FIG. 1. In particular, displays can be provided in association with either the microprocessor/transmitter node 14, or the cloud server 20, or the patient or physician mobile device 22 or the physician devices 24. All or some of these devices may have display and control capabilities.

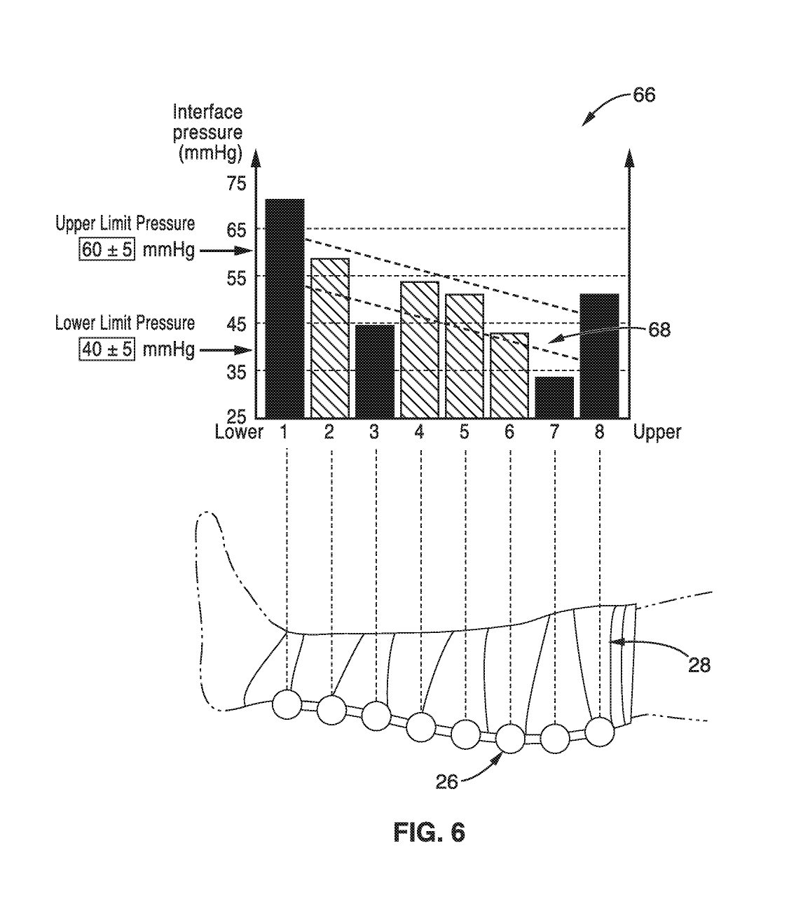

For example, the display screen show in FIG. 6 uses a numerical and graphical description of the pressure status that corresponds to the a sensor array of eight (8) sensors 26 placed at regular locations along the leg of a patient and associated with a compression device 28. The display 66 indicates the sensor pressure graphically displaying the pressure registered at each of the pressure sensors 26.

Pressure sensors 26 preferably have an operating pressure range of between 0 mmHg to 100 mmHg. Target interface pressures typically range from between 20 mmHg to 65 mmHg depending on the nature of the vascular deficiency or symptom that is being treated. Compression devices that are applied to the appendage can be adjusted to target interface pressure range between 0 mmHg to 100 mmHg using the sensor system. While this applied pressure range encompasses most pressure treatments, the sensitivity of the sensors 26 that are used can be selected to detect the upper limit of interface pressure range that is >100 mmHg.

The applied pressure of the initial application of the compression device 28 can be determined to be within a therapeutic interface pressure range and monitored over time. Applied pressure during activities such as walking can also be identified. Deviations from the set range of applied pressure will signal the need for an adjustment of the compression device to the target interface pressure range. This will avoid damage due to excessive pressure as well as insure sufficient pressure is applied to provide a therapeutic dose.

A designated threshold range of pressure values 68 is also displayed and the actual pressure registered by each sensor 26 can be compared with the threshold range of target pressures. The location of needed adjustments to the compression device 28 can also be quickly identified with reference to the display 66 so that the bandages or hook and loop fastener strips or other elements of the compression device 28 can be tightened or loosened. The changes in applied pressure at different locations from the adjustments can also be verified.

A temporal component to pressure treatment can also be introduced into potential treatment schemes. For example, sequences of applied pressures and time periods can be performed as part of the treatment. Cycles of high pressure for one duration followed by the application of lower pressure for a second duration can be applied. This may allow pressure treatments to be conducted on patients that would not normally be candidates for pressure treatments such as those with arterial issues. The timed application and release of suitable pressures can increase venous flow without aggravating the disqualifying condition.

Furthermore, one pressure can be to one location and a different pressure applied to a second location along the same appendage. For example, a graded application of increasing pressures going up or down the appendage can be verified. Also a higher pressure can be applied to one point of the appendage such as an ulcer, while a lower pressure is applied to the rest of the appendage.

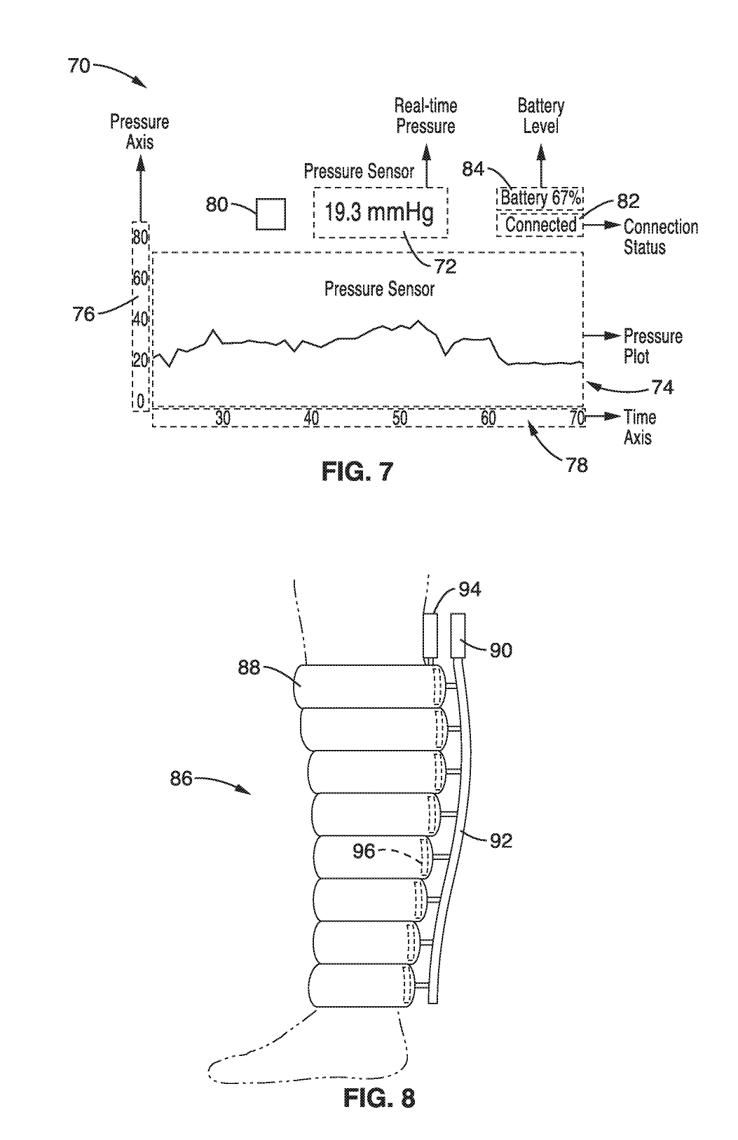

The history of applied pressure of each sensor over time and the current applied pressure can also be displayed as illustrated in FIG. 7. In this embodiment, the display 70 presents the current pressure readings 72 of an individual pressure sensor as well as a pressure plot 74 for the sensor. The scales of the pressure plot 74 are a vertical pressure axis 76 horizontal time axis 78. The display 70 also has a status indicator/alarm 80 that activates with a change in color or audible alarm when the pressure falls outside of a designated threshold indicating an adjustment to the compression device is needed. The display 70 also has an indicator 82 of whether the device is connected to a wireless network and a battery level indicator 84 for power for the sensor and node.

In addition to processing and displaying interface pressure sensing results, the system can make a muscle contractility analysis from the sensor data. For example, the number of muscle contractions over time and the duration of each muscle group contraction as well as the intensity of contraction can be demonstrated. This analysis provides more quantitative data to patient/athlete for the assessment of exercise performance or rehabilitation progress. Moreover, exact calorie count based on the number of muscle contractions, duration of each contraction and intensity of each contraction can also be calculated.

The system can also perform an interface pressure sensing analysis of the compression device. When the sensors detect interface pressures that are a designated amount below a compression garment rating or initial bandage pressure, then a loss of elasticity from the compression product or bandage is demonstrated. In one embodiment, a trigger from the device would be activated and alert notification would be sent wirelessly to the patient, MD, and caregiver that the compression product no longer provides any therapeutic value. A new device or MD order may be warranted.

The sensor data processing can take place with programming in the cloud server 20, the physician devices 24 the patient mobile device 22 or the node 14 and displayed on displays associated with these devices. However, the processed data is preferably transmitted to the cloud or clinic server 20 and automatically stored in a patient file.

The displays shown in FIG. 6 and FIG. 7 illustrate useful ways to present sensor data for analysis and recording. In other embodiments, the mobile devices, server or physician devices can also have a control function with control programming and the device displays can serve as an interface with the system components.

In one embodiment, the device programming can both automatically process and display the pressure data as well as direct wireless transmission of data to other terminal devices such as desktop computer, laptop computer, smartphone, tablet, or watch etc. The data transmitted to terminal device can be saved locally and/or upload to cloud base storage.

For example, the system conducts multi-thread receiving, parsing, processing, and rendering real-time data to users. During the initial setup of the user interface, the clinician and/or the patient predefine the optimal interface pressure range and program it accordingly. The computer program using this predefined pressure range automatically calculates the targeted pressure distribution along the limb, thus truly customizing compression therapy. As the patient gradually applies the bandage onto the limb, the embedded pressure sensors will provide a clear readout of the interface pressure and wirelessly transmit data to a mobile device. FIG. 6 illustrates such a graphic user interface design for the wearable sensors in which the pressure columns (Y axis) indicate the corresponding readings from the wearable sensing units (X axis). When the applied pressure is either lower or higher than the targeted pressure range, the indicating bars will stay red. Once the interface pressure falls in the preset range, the corresponding pressure bar will turn into green, for example. This wireless user interface enables management and monitoring of the compression therapy by patients at home or caregivers in a nursing facility with high accuracy, high compliance and high efficiency.

The control programming of the devices in the system can also allow for remote human or machine control over the compression devices and other system components. Remote monitoring and control of a compression device by a physician is illustrated with an inflatable cuff or sleeve 86 in the embodiment of FIG. 8. The lower leg is inserted into the interior of sleeve 86 in this illustration. The sleeve has a number of inflatable chambers 88 that can be filled with air or with a liquid and the filled chambers 88 apply pressure to the appendage in the interior of the sleeve. The individual chambers 88 are coupled to an inflation control 90 through a network of ducts 92. Inflation control 90 is configured to selectively inflate each chamber 88 at a time or it can inflate all of the chambers 88 to the same pressure.

The sleeve 86 also has data collection/transmitter node 94 and a set of sensors 96 disposed in the interior of the sleeve 86. The sensors 96 can either be attached to the sleeve 86 or applied directly to the skin and remain separate from the sleeve 86. The data/transmitter node 94 is also operably coupled to the inflation control 90 so that it can be controlled remotely through the transmitter of node 94.

In this embodiment, the sleeve chambers 88 are inflated by the inflation control 90 to a designated interface pressure as sensed by sensors 96 in real time confirming the actual pressure levels exerted by the sleeve 86. The pressure is monitored over time by the data/transmitter node 94. The sensor system will also verify that the sleeve is currently on the patient and can provide a pressure level and duration treatment history.

The system can control and modify function of the compression system (i.e. compression pump, boot, etc) remotely. The programming can also set the operating interface pressure range for the automated compression system i.e. when the interface pressure is below or above the pre-set range, the automated compression system will self adjust so the interface pressure stays within the pre-set range. It can also remotely control the automated compression system to actively turn it on, off, or adjust the compression mechanism to reach a target interface pressure range.

In one embodiment, sensor-data transmission node 94 and the inflation control 90 of the sleeve 86 are operably linked so that control over the inflation of each chamber 88 can be controlled by programming on a remote computer such as the mobile device 22, cloud or clinic server 20 or the physician device 24 of FIG. 1. In this embodiment, the control programming successively inflates the chambers starting from the bottom or top of the sleeve 86 to a selected pressure and then reduces the pressure to a lower pressure creating a peristaltic like wave of pressure on the appendage of the patient. The length of time of each inflation and deflation, the pressure exerted at each level and the rate of wave propagation can be determined by the system. Changes in these parameters and the initiation of the treatment can be conducted by a remote physician. The principle of peristaltic wave pressures can be applied for treatment of venous, lymphatic and even arterial diseases.

The technology described herein may be better understood with reference to the accompanying examples, which are intended for purposes of illustration only and should not be construed as in any sense limiting the scope of the technology described herein as defined in the claims appended hereto.

### EXAMPLE 1

In order to demonstrate the technology, a system was designed based on a single ionic gel-based pressure sensor of the design depicted schematically in FIG. 2B. The sensor used a nanoliter ionic gel droplet sandwiched between the top and bottom flexible ITO coated polyethylene terephthalate (PET) membranes. A separation layer supports the spacing between the two sensing membranes and on one side of the membrane, a 10 μm-high, 200 μm-diameter micropillar perform as the anchor for the ionic gel. Upon the ionic gel-electrode contact, electrical double layer (EDL) capacitance has formed. Under external mechanical loads, the polymer membranes deform, leading to the circumferential expansion of the ionic gel droplet. The variation in the ionic gel and electrode contact area will lead to a proportional change in the interfacial capacitance. To facilitate pressure distribution measurement, a 1 by 8 sensing array with 3 cm interval was designed and fabricated to achieve a pressure distribution on adult lower limb with total 25 cm device length. To match the desired pressure range (0-10 kPa) for the interface pressure measurement, each unit has a circular sensing area with 5 mm in diameter.

The fabrication process started with a 125 μm-thick transparent PET film with ITO coating (Sigma Aldrich), which was laser-machined (VersaLaser, Universal Laser) into the designated geometries as the bottom membranes of the device as shown in FIG. 2B. Then, a layer of 50 μm adhesive transfer tape (3M 467MP) is also trimmed into ‘C’ shape and laminate on to the bottom membrane. Several circular shaped PET/ITO films were consecutively cut by laser to form the top membrane. After the completion of electrode cutting, the polymer micropillar of 10 μm in height was constructed on the electrode surfaces by laminating a negative dry-film photoresist (PerMX3010, DuPont) onto the bottom membrane, photolithography, and develop the photoresist. Prepare the ionic gel by mixing Ionic liquid (1-Ethyl-3-methylimidazolium tricyanomethanide, lolitec), polyethylene glycol (PEG) 4000 (Sigma-Ardrich) and photo-initiator in 5:1:1 volume ratio. Using a microfluidic impact printing technique, nanoliter droplets (˜7 nL) of the liquid mixture will be sequentially deposited onto the micropillar, subsequently by a UV exposure for 20 s (365 nm, 22 mJ cm⁻², ABM, Inc.). After the liquid mixture cross-linked into ionic gel, the two electrode membranes were aligned and subsequently packaged along the perimeter of each unit.

The EDL capacitance (10 μF/cm²) of the sensor device was tested and found to be more than 1,000 times greater than that of solid-state counterparts at the same dimensions, demonstrating the unprecedented sensitivity of the device.

### EXAMPLE 2

In order to further demonstrate the technology, a low-power wireless interface for pressure data acquisition and processing of the microfluidic sensing array was constructed. The system provided an analog front, a microcontroller and a Bluetooth transmission module as well as graphic user interface. Analog-front component converted sensor impedance into a voltage signal. The ultra-low-power MSP430 microcontroller allowed all digital processing, including data acquisition, processing and serial communication, from which the Bluetooth transmission module can be used to achieve wireless communication with PC, tablet, cellular phone user interface or any other mobile platform.

The analog front was devised to interrogate each capacitive sensing element of the microfluidic sensing array consecutively and the collective interface pressure data was acquired into an electronic circuitry. The interfacial EDL capacitance was found to offer both stable high unit-area capacitance and minimal temperature dependence in the frequency range between 1 kHz to 20 kHz, whereas a higher operation frequency permits a higher scanning rate. Given an array of 1×8 microfluidic sensors as shown in FIG. 6, the analog front provided a frame rate up to 625 Hz.

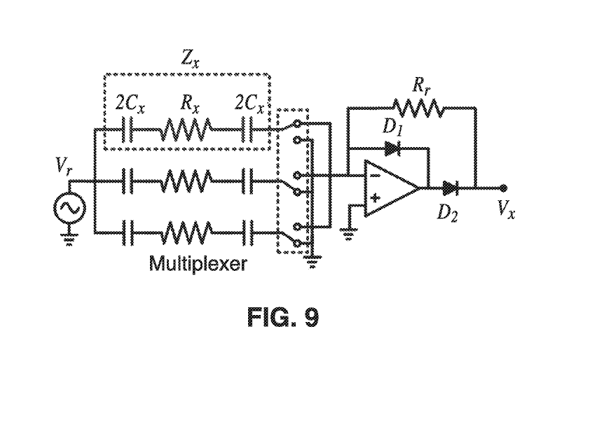

Readout circuitry with wireless communication module (Bluetooth) was developed to achieve data acquisition from the sensing array and wireless transmission. A schematic of the acquisition circuitry is shown in FIG. 9. All the sensing units were driven under a common AC input (Vᵣ). To address an individual sensing unit, the corresponding multiplexer can be activated by the micro-controller, from which h an output voltage (Vₓ) was generated, amplified and conditioned accordingly. The output voltage thus follows a linear relationship with the corresponding Impedance value (Zₓ) in the measuring unit, which can be expressed as:

$${V}_{x} = - {V}_{r} \frac{{Z}_{x}}{{R}_{r}}$$

where Rᵣ represents for a reference resistor in the amplification circuitry. In addition, two diodes (D₁ and D₂) establish a half-wave precision rectification circuit. Specifically, the input sine wave controlled by microcontroller via SPI protocol is generated by a Direct Digital Synthesis (DDS) chip, AD9833, to achieve synchronization between the sine wave and the Analog to Digital Convertor (ADC). The rectified voltage output from the inverting amplifier sampled by the ADC on a MSP430 microcontroller. The microcontroller working under 20 MHz main clock has a 12-bit ADC and supports UART and SPI protocol. As the multiplexer repeatedly scans through each sensor, the impedance value of the corresponding unit is acquired by ADC. The Bluetooth module connected with the microcontroller transmits the data via customized multi-channel protocol to the user interface on the mobile devices.

From the description herein, it will be appreciated that that the present disclosure encompasses multiple embodiments which include, but are not limited to, the following:

1. A telemedical interface pressure monitoring system, comprising: (a) a compression therapy device, the device capable of exerting an interface pressure when applied to the body of a user; (b) a sensor array with at least one pressure sensor configured to be disposed between the compression therapy device and the body of a user, the array producing sensor array signals; (c) a data collection transmission node operably coupled to the sensors of the sensor array configured to receive the sensor array signals, the node comprising a microprocessor and one or more transmitters; and (d) a data transmission receiver; (e) wherein the sensor array signals received by the node are transmitted to the data transmission receiver; and (f) wherein an interface pressure quantity is formulated from the sensor array signals.

2. The system of any preceding embodiment, wherein the compression therapy device is a device selected from the group of devices consisting of a compression bandage; a compression garment, a pneumatic sleeve, a soft cast, a hard cast and a splint.

3. The system of any preceding embodiment, wherein the pressure sensors of the sensor array comprise a sensor selected from the group of a force sensitive rubber (FSR) based pressure sensor, a conductive ink based pressure sensor, a conductive polymer based capacitive pressure sensor and a microfluidic based pressure sensor.

4. The system of any preceding embodiment, wherein the at least one transmitter is a transmitter selected from the group of Bluetooth, Wi-Fi, radio, 1G, 2G, 3G, 4G, RFID and Near Field Communication, alone or in combination.

5. The system of any preceding embodiment, further comprising at least one sensor selected from the group of sensors consisting of a temperature sensor, a heartbeat sensor, a moisture sensor and a chemical detection sensor.

6. The system of any preceding embodiment, wherein the data transmission receiver further comprises a display.

7. The system of any preceding embodiment, wherein the data collection transmission node further comprises a receiver.

8. A telemedical interface pressure monitoring system, comprising: (a) a computer server with a communications hub; and (b) a network of individual patient pressure treatment platforms configured to communicate with the computer server through the communications hub, the treatment platform comprising: (i) a compression therapy device, the device capable of exerting an interface pressure when applied to the body of a user; (ii) a sensor array with at least one pressure sensor configured to be disposed between the compression therapy device and the body of a user, the array producing sensor array signals; and (iii) a data collection transmission node operably coupled to the sensors of the sensor array configured to receive the sensor array signals, the node comprising a microprocessor, a receiver and one or more transmitters, the transmitters in communication with the communications hub of the computer server; (e) wherein the sensor array signals received by the node are transmitted to the computer server; and (f) wherein an interface pressure quantity is formulated from the sensor array signals and recorded in memory of the computer server.

9. The system of any preceding embodiment, the system further comprising: a controller with an interface and display configured to control the computer server and to display sensor data.

10. The system of any preceding embodiment, the system further comprising: a mobile device with an interface and display configured to communicate with the computer server and to display sensor data.

11. The system of any preceding embodiment, wherein the compression therapy device is a device selected from the group of devices consisting of a compression bandage; a compression garment, a soft cast, a hard cast and a splint.

12. The system of any preceding embodiment, wherein the pressure sensors of the sensor array comprise a sensor selected from the group of a force sensitive rubber (FSR) based pressure sensor, a conductive ink based pressure sensor, a conductive polymer based capacitive pressure sensor and a microfluidic based pressure sensor.

13. The system of any preceding embodiment, wherein the at least one transmitter is a transmitter selected from the group of Bluetooth, Wi-Fi, radio, 1G, 2G, 3G, 4G, RFID and Near Field Communication, alone or in combination.

14. The system of any preceding embodiment, the treatment platform further comprising at least one sensor selected from the group of sensors consisting of a temperature sensor, a heartbeat sensor, a moisture sensor and a chemical detection sensors.

15. A telemedical interface pressure monitoring system, comprising: (a) a compression therapy device capable of exerting an interface pressure when applied to the body of a user, the device comprising: (i) an inflatable sleeve with at least one inflatable chamber; (ii) an inflator fluidly coupled to the sleeve configured to inflate each inflatable chamber with a volume of fluid; and (iii) an inflation controller configured to control the inflation of the chambers of the sleeve; (b) a sensor array with at least one pressure sensor configured to be disposed on a surface of the sleeve, the array producing sensor array signals; (c) a data collection transmission node operably coupled the inflation controller and to the sensors of the sensor array configured to receive the sensor array signals, the node comprising a microprocessor and one or more transmitters; and (d) a computer processor operably coupled to the transmission node with a memory storing instructions executable on the computer processor, wherein when executed by the computer processor the instructions perform steps comprising: (i) receiving sensor data transmitted from the transmission node; (ii) determining an interface pressure; and (iii) controlling the inflation controller to inflate the inflatable sleeve to a designated pressure.

16. The system of any preceding embodiment, wherein the instructions further comprise recording interface pressures over time.

17. The system of any preceding embodiment, the computer processor further comprising an interface, wherein a sleeve inflatable chamber pressure can be designated and the inflation controller controlled remotely.

18. The system of any preceding embodiment, wherein the interface further comprises a display of interface pressure data.

19. The system of any preceding embodiment, wherein the at least one transmitter is a transmitter selected from the group of Bluetooth, Wi-Fi, radio, 1G, 2G, 3G, 4G, RFID and Near Field Communication, alone or in combination.

20. The system of any preceding embodiment, the sensor array further comprising at least one sensor selected from the group of sensors consisting of a temperature sensor, a heartbeat sensor, a moisture sensor and a chemical detection sensors.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

Embodiments of the present technology may be described with reference to flowchart illustrations of methods and systems, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that the terms “programming” or “program executable” as used herein refer to one or more instructions that can be executed by a processor to perform a function as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors. It will further be appreciated that as used herein, that the terms processor, computer processor, central processing unit (CPU), and computer are used synonymously to denote a device capable of executing the instructions and communicating with input/output interfaces and/or peripheral devices.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A telemedical interface pressure monitoring system, comprising:
(a) a compression therapy device, said device capable of exerting an interface pressure when applied to the body of a user;
(b) a sensor array with at least one pressure sensor configured to be disposed between the compression therapy device and the body of a user, said array producing sensor array signals;
(c) a data collection transmission node operably coupled to the sensors of the sensor array configured to receive the sensor array signals, said node comprising a microprocessor and one or more transmitters; and
(d) a data transmission receiver;
(e) wherein said sensor array signals received by the node are transmitted to said data transmission receiver; and
(f) wherein an interface pressure quantity is formulated from said sensor array signals.

2. A system as recited in claim 1, wherein said compression therapy device is a device selected from the group of devices consisting of a compression bandage; a compression garment, pneumatic sleeve, a soft cast, a hard cast and a splint.

3. A system as recited in claim 1, wherein said pressure sensors of the sensor array comprise a sensor selected from the group of a force sensitive rubber (FSR) based pressure sensor, a conductive ink based pressure sensor, a conductive polymer based capacitive pressure sensor and a microfluidic based pressure sensor.

4. A system as recited in claim 1, wherein said at least one transmitter is a transmitter selected from the group of Bluetooth, Wi-Fi, radio, 1G, 2G, 3G, 4G, RFID and Near Field Communication, alone or in combination.

5. A system as recited in claim 1, further comprising at least one sensor selected from the group of sensors consisting of a temperature sensor, a heartbeat sensor, a moisture sensor and a chemical detection sensor.

6. A system as recited in claim 1, wherein said data transmission receiver further comprises a display.

7. A system as recited in claim 1, wherein said data collection transmission node further comprises a receiver.

8. A telemedical interface pressure monitoring system, comprising:
(a) a computer server with a communications hub; and
(b) a network of individual patient pressure treatment platforms configured to communicate with the computer server through the communications hub, said treatment platform comprising:
(i) a compression therapy device, said device capable of exerting an interface pressure when applied to the body of a user;
(ii) a sensor array with at least one pressure sensor configured to be disposed between the compression therapy device and the body of a user, said array producing sensor array signals; and
(iii) a data collection transmission node operably coupled to the sensors of the sensor array configured to receive the sensor array signals, said node comprising a microprocessor, a receiver and one or more transmitters, said transmitters in communication with the communications hub of the computer server;
(e) wherein said sensor array signals received by the node are transmitted to said computer server; and
(f) wherein an interface pressure quantity is formulated from said sensor array signals and recorded in memory of said computer server.

9. A system as recited in claim 8, said system further comprising:
a controller with an interface and display configured to control said computer server and to display sensor data.

10. A system as recited in claim 8, said system further comprising:
a mobile device with an interface and display configured to communicate with said computer server and to display sensor data.

11. A system as recited in claim 8, wherein said compression therapy device is a device selected from the group of devices consisting of a compression bandage; a compression garment, a soft cast, a hard cast and a splint.

12. A system as recited in claim 8, wherein said pressure sensors of the sensor array comprise a sensor selected from the group of a force sensitive rubber (FSR) based pressure sensor, a conductive ink based pressure sensor, a conductive polymer based capacitive pressure sensor and a microfluidic based pressure sensor.

13. A system as recited in claim 8, wherein said at least one transmitter is a transmitter selected from the group of Bluetooth, Wi-Fi, radio, 1G, 2G, 3G, 4G, RFID and Near Field Communication, alone or in combination.

14. A system as recited in claim 8, said treatment platform further comprising at least one sensor selected from the group of sensors consisting of a temperature sensor, a heartbeat sensor, a moisture sensor and a chemical detection sensors.

15. A telemedical interface pressure monitoring system, comprising:
(a) a compression therapy device capable of exerting an interface pressure when applied to the body of a user, said device comprising:
(i) an inflatable sleeve with at least one inflatable chamber;
(ii) an inflator fluidly coupled to the sleeve configured to inflate each inflatable chamber with a volume of fluid; and
(iii) an inflation controller configured to control the inflation of the chambers of the sleeve;
(b) a sensor array with at least one pressure sensor configured to be disposed on a surface of said sleeve, said array producing sensor array signals;
(c) a data collection transmission node operably coupled said inflation controller and to the sensors of the sensor array configured to receive the sensor array signals, said node comprising a microprocessor and one or more transmitters; and
(d) a computer processor operably coupled to the transmission node with a memory storing instructions executable on the computer processor, wherein when executed by the computer processor said instructions perform steps comprising:
(i) receiving sensor data transmitted from said transmission node;
(ii) determining an interface pressure; and
(iii) controlling the inflation controller to inflate the inflatable sleeve to a designated pressure.

16. The system as recited in claim 15, wherein said instructions further comprise recording interface pressures over time.

17. A system as recited in claim 15, said computer processor further comprising an interface, wherein a sleeve inflatable chamber pressure can be designated and the inflation controller controlled remotely.

18. A system as recited in claim 17, wherein said interface further comprises a display of interface pressure data.

19. A system as recited in claim 15, wherein said at least one transmitter is a transmitter selected from the group of Bluetooth, Wi-Fi, radio, 1G, 2G, 3G, 4G, RFID and Near Field Communication, alone or in combination.

20. A system as recited in claim 15, said sensor array further comprising at least one sensor selected from the group of sensors consisting of a temperature sensor, a heartbeat sensor, a moisture sensor and a chemical detection sensors.
